(19) United States
(12) Patent Application Publication
(10) Pub. No.: US 2025/0312560 A1
(43) Pub. Date: Oct. 9, 2025

(54) VIBRATION GENERATION DEVICE FOR IMPROVING SLEEP

(71) Applicant: BRLAB INC., Seoul (KR)

(72) Inventors: Jongmin LEE, Seoul (KR); Hyunbin KWON, Seoul (KR); Kwangsuk PARK, Seoul (KR); Byunghun CHOI, Seoul (KR); Jonghyeok JEONG, Seoul (KR); Ilsong LEE, Seoul (KR)

(21) Appl. No.: 19/245,019

(22) Filed: Jun. 20, 2025

Related U.S. Application Data
(63) Continuation of application No. PCT/KR2023/021425, filed on Dec. 22, 2023.

(30) Foreign Application Priority Data
Dec. 22, 2022 (KR) 10-2022-0182003
Dec. 22, 2022 (KR) 10-2022-0182004
Apr. 10, 2023 (KR) 10-2023-0047030

(51) Int. Cl. A61M 21/02; A61M 21/00

(52) U.S. Cl. CPC A61M 21/02; A61M 2021/0022

(57) ABSTRACT

The present disclosure relates to a sleep-inducing vibration device that provides vibratory stimulation to a user to induce sleep. The vibration generation device 100 for improving sleep in the present disclosure may include a vibration plate 110 provided inside a mattress M, an actuator 120 provided below the vibration plate to apply vibration to the vibration plate, and a vibration transmitting body 130 provided between the vibration plate and the actuator to transmit vibration generated from the actuator to the vibration plate.

### CROSS-REFERENCE TO RELATED APPLICATIONS

This application is a Continuation of Application No. PCT/KR2023/021425, filed on Dec. 22, 2023, which in turn claims the benefit of Korean Patent Applications No. 10-2022-0182003, filed on Dec. 22, 2022, No. 10-2022-0182004, filed on Dec. 22, 2022, and No. 10-2023-0047030, filed on Apr. 10, 2023. The entire disclosures of all these applications are hereby incorporated by reference.

### TECHNICAL FIELD

The present disclosure relates to a vibration generation device for improving sleep that provides vibratory stimulation to a user to induce sleep, and more particularly, to a vibration generation device that can be provided in a layer such as a mattress or mat on which a user can lie down and sleep to generate vibration toward a lying user so as to induce a desired sleep state.

### BACKGROUND ART

Sleep is a process of removing fatigue substances from the brain and has a significant impact on mental health. In particular, if someone does not get enough sleep for a long period of time, toxic proteins are not discharged from the brain, which not only makes it difficult to carry out daily life the next day, but also causes decreased concentration, frequent accidents, decreased motivation, daytime sleepiness, and fatigue. Furthermore, if the insomnia persists, the risk of dementia increases and it can even develop into depression and anxiety.

Recently, in order to improve the quality of sleep, methods such as playing quiet music or white noise around him or her while sleeping are being explored, but they are only partially helpful in improving a user's sleep quality, and the effect does not meet expectations.

The user's sleep state is ultimately determined by how much and how the sympathetic or parasympathetic nerves are activated/deactivated, and an aspect of the present disclosure is to induce the user's desired sleep state through vibration by providing a vibration generation device below the position where the user is lying.

### DISCLOSURE OF INVENTION

### Technical Problem

The present disclosure is contrived to solve the foregoing problems, and an aspect of the present disclosure is to provide a sleep-inducing vibration device that enables a person to have a deep sleep without relying on external noise.

Technical problems of the present disclosure are not limited to the above-mentioned problems, and other technical problems which are not mentioned herein will be clearly understood by those skilled in the art from the description below.

### Technical Solution

A vibration generation device for improving sleep in the present disclosure may include a vibration plate provided inside a mattress, an actuator provided below the vibration plate to apply vibration to the vibration plate, and a vibration transmitting body provided between the vibration plate and the actuator to transmit vibration generated from the actuator to the vibration plate.

In addition, the vibration plate may be made of a flexible material.

Moreover, the actuator may include a lower body, a cylindrical bobbin disposed in the center of the lower body, a coil wound around the bobbin to move up and down along the bobbin, a magnet provided above the lower body to surround the bobbin, an upper body coupled to an upper part of the lower body while accommodating the bobbin and the magnet therein, and a current signal input terminal disposed on one side of the upper body to apply a current to the coil.

In this case, the vibration transmitting body may have a through hole disposed in the center, wherein a fitting projection that fits into the through hole is disposed in the center of the upper body.

In addition, the vibration transmitting body may be provided with an upper surface that is in close contact with a bottom surface of the vibration plate, a lower surface that is in close contact with an upper end of the actuator, and a side surface that connects the upper surface and the lower surface while surrounding the fitting projection.

Moreover, the vibration plate, the actuator, and the vibration transmitting body may be integrally coupled by bolts and nuts.

In this case, a plurality of protruding portions may be disposed on an outside of the actuator, wherein close contact protrusions that are in close contact with the protruding portions are disposed on a lower surface of the vibration transmitting body, and bolt fitting holes are positioned on the same vertical line in the vibration plate, the protruding portion of the actuator, and the upper surface and the close contact protrusion of the vibration transmitting body, respectively.

Meanwhile, one side section corresponding to the current signal input terminal may preferably be disposed to be open.

### Advantageous Effects

According to a vibration generation device for improving sleep in the present disclosure as described above, since an actuator that induces vibration is provided inside a mattress, unlike in the past when it depended only on external noise, when a user lying on the mattress is stimulated by vibration, the user's sympathetic or parasympathetic nerves may be activated in a direction that improves the quality of sleep so as to allow the user to easily get a deep sleep.

In addition, since the present disclosure is provided with an actuator that generates vibration, a vibration plate that transmits the vibration to a user, and a vibration transmitting body that transmits the vibration of the actuator to the vibration plate, the vibration efficiency may be greatly improved, and the volume and area of the vibration transmitting body may be changed so as to have an effect of easily and freely adjusting a range of vibration transmitted to the user.

The effects of the present disclosure are not limited to those mentioned above, and may be clearly understood by those skilled in the art from the description throughout the specification, but also include other effects that are not explicitly mentioned.

### BRIEF DESCRIPTION OF THE DRAWINGS

FIG. 1 shows a cross-sectional view of a vibration generation device for improving sleep in a state of use according to the present disclosure.

FIG. 2 shows a bottom perspective view of a vibration generation device for improving sleep according to the present disclosure.

FIG. 3 shows an exploded perspective view of a vibration generation device for improving sleep according to the present disclosure.

FIG. 4 shows a cross-sectional view of a vibration generation device for improving sleep according to the present disclosure.

### BEST MODE FOR CARRYING OUT THE INVENTION

Hereinafter, preferred embodiments of the present disclosure will be described in detail with reference to the accompanying drawings. Advantages and features of the present disclosure, and methods of accomplishing the same will be clearly understood with reference to the following embodiments described below in detail in conjunction with the accompanying drawings. However, the present disclosure is not limited to those embodiments disclosed below but may be implemented in various different forms, and it should be noted that the present embodiments are merely provided to make a full disclosure of the present invention and also to allow those skilled in the art to know the full range of the disclosure, and therefore, the present disclosure is to be defined only by the scope of the appended claims. Like reference numerals refer to like elements throughout the specification.

Unless otherwise defined, all terms (including technical and scientific terms) used herein may be used with meanings that can be commonly understood by those skilled in the art to which the present disclosure pertains. Additionally, terms defined in commonly used dictionaries are not interpreted ideally or excessively unless clearly specifically defined. It should be noted that the terms used herein are merely used to describe the embodiments, but not to limit the present disclosure. In this specification, unless clearly used otherwise, expressions in a singular form include a plural form.

The term “comprises” and/or “comprising” used in the specification intend to express a constituent element, a step, an operation and/or a device does not exclude the existence or addition of one or more other constituent elements, steps, operations and/or devices.

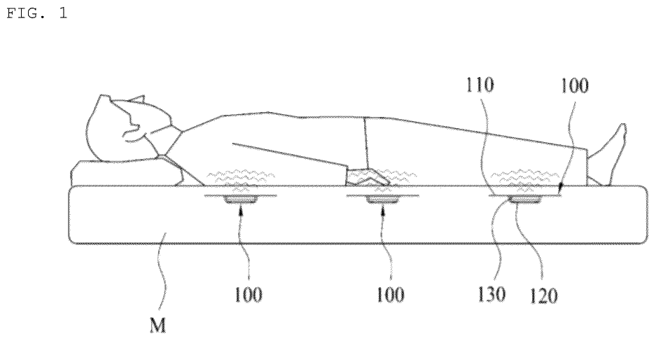

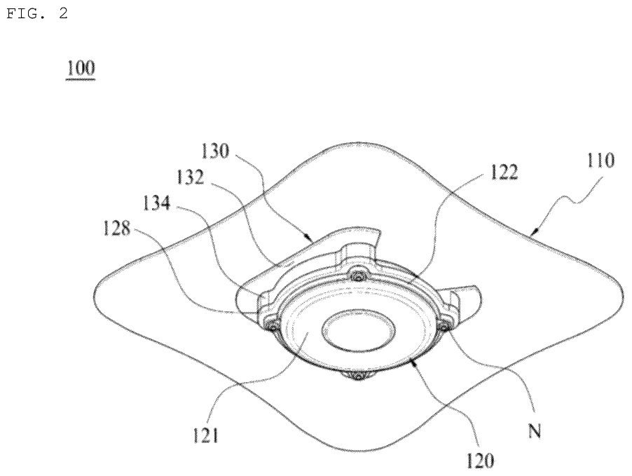

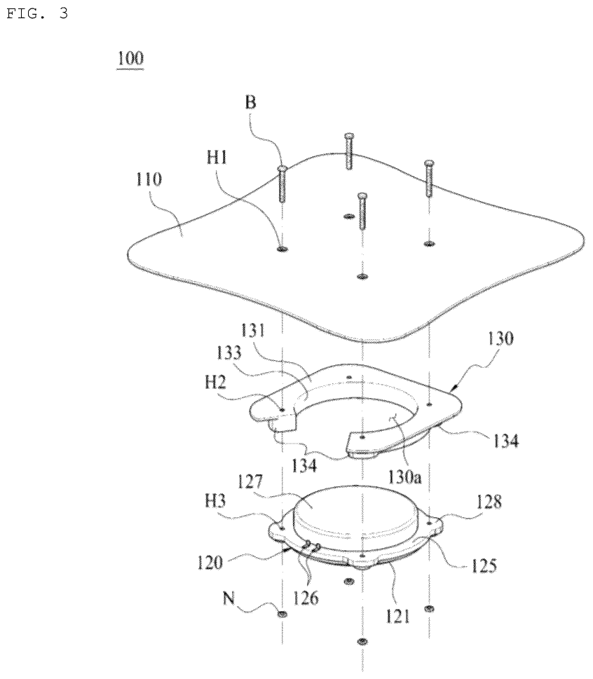

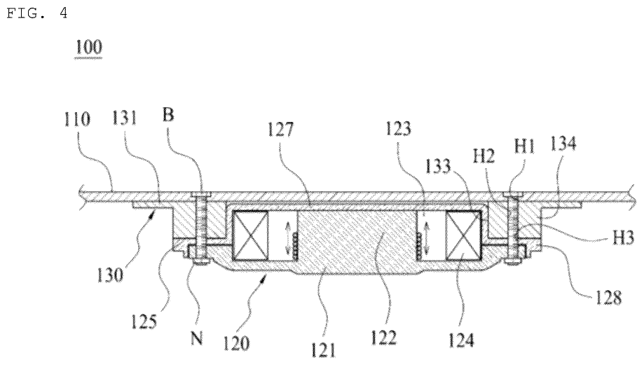

FIG. 1 is a cross-sectional view of a vibration generation device for improving sleep in a state of use according to the present disclosure, FIG. 2 is a bottom perspective view of a vibration generation device for improving sleep according to the present disclosure, FIG. 3 is an exploded perspective view of a vibration generation device for improving sleep according to the present disclosure, and FIG. 4 is a cross-sectional view of a vibration generation device for improving sleep according to the present disclosure, and as shown, a vibration generation device 100 for improving sleep according to the present disclosure includes a vibration plate 110, an actuator 120, and a vibration transmitting body 130 as main components.

First, with respect to a vibration plate 110, the vibration plate 110 is provided inside a mattress M. Here, the mattress M refers to any member on which a user can lie, sit, or lean on to rest, and there are no restrictions on its material or shape. For example, the mattress M may be made of wood or metal rather than fiber, or may be a mat that can be laid on the mattress M. A plurality of layers may be cumulatively included inside the mattress M, and the vibration generation device for improving sleep according to the present disclosure may be provided in a specific layer or between layers.

The vibration plate 110, which is a plate-shaped member provided inside the mattress, is preferably made of a flexible material so as to allow the user not to feel a foreign body sensation when lying down on the mattress M while preventing damage due to external impact.

Moreover, the vibration plate 110 must have an appropriate strength to increase vibration transmission efficiency. Considering this, the vibration plate 110 is preferably made of polyethylene (PE) material that has both flexibility and strength, but is not limited thereto.

Moreover, the vibration plate 110 may include an intermediate member with an excellent strength, such as synthetic resin, metal, or stone, and a cover portion made of a flexible material that surrounds a surface of the intermediate member.

The thickness, area, and installation location of the vibration plate 110 may of course be set in various ways according to the designer's intention.

Next, with respect to the actuator 120, the actuator 120 is configured to receive power from the outside to generate vibration, and provided below the vibration plate 110, more precisely, beneath the vibration plate 110.

The actuator 120 may be applied without limitation as long as having a structure that can generate vibration.

In one embodiment, the actuator 120 may include a lower body 121, a cylindrical bobbin 122 disposed in the center of the lower body 121, a coil 123 wound around the bobbin 122 to move up and down along the bobbin 122, a magnet 124 provided above the lower body 121 to surround the bobbin 122, an upper body 125 coupled to an upper part of the lower body 121 while accommodating the bobbin 122 and the magnet 124 therein, and a current signal input terminal 126 disposed on one side of the upper body 125 to apply a current to the coil 123.

The actuator 120 configured in this manner moves, when the user applies a current to the coil 123 through the current signal input terminal 126, the coil 123 up and down along the bobbin 122 due to a mutual induction between the coil 123 and the magnet 124, and as a result, vibration is generated.

Accordingly, the user may change a momentum of the coil 123 by performing an operation (any operation that can be performed via an operating panel for controlling the vibration generation device) so as to adjust an intensity of the current applied to the coil 123, thereby freely adjusting the vibration intensity.

Although not shown, a control module that adjusts an intensity of the current applied to the coil 123 may be connected to the current signal input terminal 126. The control module may be preferably configured to apply an independent current signal to each actuator 120.

Next, with respect to the vibration transmitting body 130, the vibration transmitting body 130 is provided between the vibration plate 110 and the actuator 120 to serve to transmit vibration generated from the actuator 120 to the vibration plate 110.

The vibration transmitting body 130 may change the property of vibration transmitted to the vibration plate 110 depending on a volume or area of the cross-section. That is, when the volume or the area of the cross-section of the vibration transmitting body 130 is reduced, vibration is transmitted to the vibration plate 110 only for a local area, and when the volume or the area of the cross-section of the vibration transmitting body 130 is expanded, vibration is transmitted to the vibration plate 110 for a wider area as being amplified and spread. The setting of the volume or cross-sectional area of the vibration transmitting body 130 must be carefully considered in the design stage of the vibration transmitting body 130.

Meanwhile, a through hole 130a may be formed in the center of the vibration transmitting body 130, and a fitting projection 127 fitted into the through hole 130a may be disposed in the center of the upper body 125.

In this case, the vibration transmitting body 130 and the actuator 120 may be coupled very easily and simply by fitting the vibration transmitting body 130 into the actuator 120.

In the coupling structure, the vibration transmission body 130 may have an upper surface 131 that is in close contact with a lower surface of the vibration plate 110, a lower surface 132 that is in close contact with an upper end of the actuator 120, and a side surface 133 that connects the upper surface 131 and the lower surface 132 while surrounding the fitting projection 127.

In addition, the vibration transmitting body 130 preferably has one side section 135 corresponding to the current signal input terminal 126, which is disposed to be open so as not to interfere with the current signal input terminal 126.

Moreover, the vibration plate 110, the actuator 120, and the vibration transmitting body 130 are preferably integrally connected so as not to allow the vibration of the actuator 120 to leak into the mattress M during the process of being transmitted to the vibration plate 110.

In this case, the vibration plate 110, the actuator 120, and the vibration transmitting body 130 may be combined in various ways, but preferably combined, for the convenience of assembly and maintenance, so as to be assembled using separate coupling members such as bolts (B) or screws.

As such, in the case of using separate coupling members, the actuator 120 has a plurality of protruding portions 128 disposed on the outside, and close contact protrusions 134 that are in close contact with the protruding portions 128 are disposed on the lower surface 132 of the vibration transmitting body 130.

Accordingly, bolt fitting holes H1, H2, H3 positioned on the same vertical line are disposed in the vibration plate 110, the protruding portion 128 of the actuator 120, and the upper surface 131 and the close contact protrusion 134 of the vibration transmission body 130, respectively.

In this case, a worker may easily and simply couple the vibration plate 110, the actuator 120, and the vibration transmitting body 130 into an integral body by sequentially fitting a bolt B into the bolt insertion holes H1, H2, H3 from an upper part of the vibration plate 110 and then fastening it with a nut N from a lower part of the actuator 120.

At least one or more of the vibration generation devices 100 for improving sleep in the present disclosure, which are configured in this manner, are installed so as to transmit vibration to a surface of the mattress M, and the installation location, quantity, and method of fixing to the mattress M are not particularly limited.

Accordingly, when the user applies a current signal to the actuator 120 in each of the vibration generation devices 100 installed on the mattress M using a control module, the actuator 120 vibrates in response to the current signal, and the vibration is transmitted to the user lying on the mattress M through the vibration transmitting body 130 and the vibration plate 110 so as to allow the user to receive vibration stimulation to his or her body parts.

The continuous vibration stimulation may activate the user's sympathetic or parasympathetic nerves in a way that improves the quality of sleep so as to allow the user to feel comfortable and easily fall into a deep sleep. Additionally, in some cases, vibration stimulation may be generated to wake the user.

In this case, the user may fall into a deep sleep according to the vibration intensity, vibration pattern, and vibration duration previously programmed in the control module, or may individually adjust and use the respective actuators 120 with an appropriate vibration intensity, vibration pattern, and vibration duration that suits his or her condition and preference.

Meanwhile, in addition to the purpose of promoting a deep sleep, vibration stimulation may also be used to wake up the user according to an alarm setting preset by the user through the control module.

That is, the user may set the operation start time, vibration intensity, and vibration duration of the actuator 120 through the control module.

As described above, the embodiments of the present disclosure have been described with reference to the accompanying drawings, but it will be apparent to those skilled in the art to which the disclosure pertains that the disclosure can be embodied in other specific forms without departing from the concept and essential characteristics thereof. Therefore, it should be understood that embodiments described above are merely illustrative but not restrictive in all aspects.

### DESCRIPTION OF SYMBOLS

100: Vibration generation device for sleep improvement 110: Vibration plate 120: Actuator 121: Lower body
122: Bobbin 123: Coil 124: Magnet 125: Upper body 126: Current signal input terminal 127: Fitting projection 128: Protruding portion 130: Vibration transmitting body 130a: Through hole 131: Upper surface 132: Lower surface 133: Side surface 134: Close contact protrusion 135: One side section B: Bolt H1, H2, H3: Bolt fitting hole M: Mattress

1. A vibration generation device for improving sleep, the device comprising:
a vibration plate provided inside a mattress;
an actuator provided below the vibration plate to apply vibration to the vibration plate; and
a vibration transmitting body provided between the vibration plate and the actuator to transmit vibration generated from the actuator to the vibration plate.

2. The device of claim 1, wherein the vibration plate is made of a flexible material.

3. The device of claim 1, wherein the actuator comprises:
a lower body;
a cylindrical bobbin disposed in the center of the lower body;
a coil wound around the bobbin to move up and down along the bobbin;
a magnet provided above the lower body to surround the bobbin;
an upper body coupled to an upper part of the lower body while accommodating the bobbin and the magnet therein; and
a current signal input terminal disposed on one side of the upper body to apply a current to the coil.

4. The device of claim 3, wherein the vibration transmitting body has a through hole disposed in the center, and
wherein a fitting projection that fits into the through hole is disposed in the center of the upper body.

5. The device of claim 4, wherein the vibration transmitting body is provided with an upper surface that is in close contact with a upper surface of the actuator, a lower surface that is in close contact with an upper end of the actuator, and a side surface that connects the upper surface and the lower surface while surrounding the fitting projection.

6. The device of claim 5, wherein the vibration plate, the actuator, and the vibration transmitting body are integrally coupled by bolts and nuts.

7. The device of claim 6, wherein a plurality of protruding portions are disposed on an outside of the actuator,
wherein close contact protrusions that are in close contact with the protruding portions are disposed on a lower surface of the vibration transmitting body, and
wherein bolt fitting holes are positioned on the same vertical line in the vibration plate, the protruding portion of the actuator, and the upper surface and the close contact protrusion of the vibration transmitting body, respectively.

8. The device of claim 3, wherein one side section corresponding to the current signal input terminal is disposed to be open.
